(19) United States
(12) Patent Application Publication
(10) Pub. No.: US 2021/0137450 A1
(43) Pub. Date: May 13, 2021

(54) BREATH MONITORING AND FEEDBACK APPLICATION AND METHODS

(71) Applicant: Aditya Agarwal, Santa Clara, CA (US)

(72) Inventor: Aditya Agarwal, Santa Clara, CA (US)

(21) Appl. No.: 17/035,393

(22) Filed: Sep. 28, 2020

Related U.S. Application Data
(63) Continuation-in-part of application No. 15/931,407, filed on May 13, 2020, now abandoned.
(60) Provisional application No. 62/847,144, filed on May 13, 2019.

(51) Int. Cl. A61B 5/00; A61B 5/16; A61B 5/113; A61B 5/0205; A61B 5/11

(52) U.S. Cl. CPC A61B 5/486; A61B 5/165; A61B 5/1135; A61B 5/6823; A61B 5/6831; H04W 88/02; A61B 5/002; A61B 5/1116; A61B 5/7455; A61B 5/742; A61B 5/7405; A61B 5/0205

(57) ABSTRACT

In one aspect, a computerized method breath monitoring and feedback application comprising the steps of providing a breath monitoring and feedback application in a mobile device; providing a means to monitor a user breath; communicating the user breath statistics to the breath monitoring and feedback application; and with the breath monitoring and feedback application, communicating an application mediation technique to the user.

### CLAIM OF PRIORITY

This application claims priority to U.S. application Ser. No. 15/931,407, titled BREATH MONITORING AND FEEDBACK APPLICATION AND METHODS, and filed on 13 May 2020. This application is incorporated herein by reference.

U.S. application Ser. No. 15/931,407 claims priority to U.S. Provisional Application No. 62/847,144, titled BREATH MONITORING AND FEEDBACK APPLICATION AND METHODS, and filed on 13 May 2019. This application is incorporated herein by reference.

### BACKGROUND

Scientific studies have shown that controlled breathing has many benefits. Controlled breathing exercise(s) can also aid in calming the nervous system, reducing stress and stress-related conditions, lowering heart rate, lowering blood pressure, increasing alertness and boosting the immune system. With all these benefits in mind, improvements to technologies that assist a user to monitor breathe volume and provide periodic reminders are desired.

### BRIEF SUMMARY OF THE INVENTION

In one aspect, a computerized method breath monitoring and feedback application comprising the steps of providing a breath monitoring and feedback application in a mobile device; providing a means to monitor a user breath; communicating the user breath statistics to the breath monitoring and feedback application; and with the breath monitoring and feedback application, communicating an application mediation technique to the user.

### BRIEF DESCRIPTION OF THE DRAWINGS

The present application can be best understood by reference to the following description taken in conjunction with the accompanying figures, in which like parts may be referred to by like numerals.

FIG. 1 illustrates an example process for determining a user's breath state, according to some embodiments.

FIG. 2 illustrates an example process for determining a user's emotional state and providing feedback to a user, according to some embodiments.

FIG. 3 illustrates an example series of modes of a breath monitoring and feedback application, according to some embodiments.

FIG. 4 illustrate a series of example screen shots of a square-breathing mode user interface, according to some embodiments.

FIG. 5 illustrates an example system for implementing a breath monitoring and feedback device, according to some embodiments.

FIG. 6 illustrate an example images of various aspects of a breath monitoring and feedback device, according to some embodiments.

FIG. 7 illustrates an example use of a breath monitoring and feedback device on a user, according to some embodiments.

FIG. 8 depicts an exemplary computing system that can be configured to perform any one of the processes provided herein.

FIG. 9 illustrates an example time-series graph of a user's breath measurements, according to some embodiments.

The Figures described above are a representative set, and are not an exhaustive with respect to embodying the invention.

### DESCRIPTION

Disclosed are a system, method, and article of manufacture of a breath monitoring and feedback application. The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to “one embodiment,” “an embodiment,” “one example,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

Accelerometer is a device that measures proper acceleration (e.g. a or rate of change of velocity).

Application programming interface (API) can specify how software components of various systems interact with each other.

Bluetooth is a wireless technology standard for exchanging data between fixed and mobile devices over short distances using short-wavelength UHF radio waves.

Deep learning is a family of machine learning methods based on learning data representations. Learning can be supervised, semi-supervised or unsupervised.

Gyroscope is a device used for measuring or maintaining orientation and angular velocity. Example gyroscope systems include, inter alia: microchip-packaged MEMS gyroscopes found in electronic devices, solid-state ring lasers, fiber optic gyroscopes, etc.

Heart rate monitor (HRM) is a personal monitoring device that allows one to measure/display heart rate in real time or record the heart rate for later study.

Internet of things (IoT) can be the internetworking of physical devices. Said device can include, inter alia: electronics, software, actuators, and connectivity/networking systems which allows these things to connect, interact and/or exchange data.

Load cell is a transducer that is used to create an electrical signal whose magnitude is directly proportional to the force being measured.

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity and metric learning, and/or sparse dictionary learning.

Random forests (RF) (e.g. random decision forests) are an ensemble learning method for classification, regression and other tasks, that operate by constructing a multitude of decision trees at training time and outputting the class that is the mode of the classes (e.g. classification) or mean prediction (e.g. regression) of the individual trees. RFs can correct for decision trees' habit of overfitting to their training set.

Square breath pattern involves a longer holding of the breath, so that the holding period equals the breathing period (e.g. 8 beats in, 16 beats hold, 8 beats out; etc.).

Strain gauge measures strain on an object. A strain gauge can be attached to the object by a suitable adhesive (e.g. cyanoacrylate, etc.). As the object is deformed, the foil is deformed, causing its electrical resistance to change. This resistance change can be measured using a Wheatstone bridge, and the like. The resistance change can be is related to the strain by a quantity known as the gauge factor.

Virtual reality (VR) is an takes place within a computer-generated reality of immersive environments can be similar to and/or completely different from the real world.

Exemplary Methods

A breath monitoring and feedback application is provided. The breath monitoring and feedback application can use various user-worn sensor systems to monitor a user's breath rate. The breath monitoring and feedback application can include and/or be coupled with other biosensors (e.g. heart-rate monitors, user posture sensors, user motion sensors, microphones, galvanic skin response sensors, etc.). The breath monitoring and feedback application can combine this information in various forms to determine a current user emotional state. The breath monitoring and feedback application can include an application instance in a user mobile device (e.g. a smart phone, tablet computer, smart watch, head-mounted display, etc.). The mobile-device application can be used to provide feedback to the user. Example feedback can include user breath and/or other biosensor information (e.g. can be displayed, audio queues, haptic codes/alerts, etc.). The breath monitoring and feedback application can store user historical data in a data store. This data can be analyzed and opportunities for improvement can be determined. Machine-learning methods can be applied to train the historical data set and/or rank improvement advice. This information can also be provided to the user to improve the user's meditation and other mindfulness training experiences. Various methods and systems for implementing a breath monitoring and feedback application are now provided.

FIG. 1 illustrates an example process 100 for determining a user's breath state, according to some embodiments. In step 102, process 100 can attach a strain gauge to a load cell. In step 104, process 100 can strap strain gauge and load cell around a user's diaphragm. In step 106, process 100 can calibrate loadcell using the circumference of the diaphragm area at the resting state. In step 108, process 100 can as user breaths in and out, measure displacement forces on load cell. In step 110, process 100 can correlate output of step 108 to specified breathing stats (e.g. normal, shallow, deep breathing, etc.).

FIG. 2 illustrates an example process 200 for determining a user's emotional state and providing feedback to a user, according to some embodiments. In step 202, process 200 can provide current user breathing states. In step 204, process 200 can use accelerometer and/or gyroscope data to estimate user posture. In step 206, process 200 can obtain a user heart rate from heart-rate monitor. In step 208, process 200 can estimate a current user emotional state from outputs of steps 202-206. In step 210, process 200 can with a companion mobile-device application, provide user feedback in real-time and/or historical views of past emotional states. It is noted that breath monitoring and feedback application can be used periodically throughout the day. Haptic feedback can be utilized to remind the user to take deep controlled breaths to be more aware of his surroundings. For example, the haptic feedback system (e.g. includes haptic motors 614, etc.) can be integrated into the undergarments (e.g. women's sports bra, in men's undershirts, etc.).

FIG. 3 illustrates an example series of modes 302-310 of a breath monitoring and feedback application, according to some embodiments. An example of bringing the mind to present mode 302 can utilize a haptic motor on the breath-monitoring device to remind the in a timely manner to take deep breaths and maintain proper posture (e.g. during a meditation session, while performing a specified activity, etc.). Bring the mind to present mode 302 can be used to return the mind to present and improve the overall mental health of the user. Bring the mind to present mode 302 can use processes 100 and 200 to detect when the user is in a stressed emotional state. Bring the mind to present mode 302 can include utilize a recommendation engine and the mobile-device application to recommend breathing exercise to reduce stress. These breathing exercises can be tied to the user's current context and/or activity.

A breathing exercise mode 306 can be implemented. Users can perform breathing exercises using a companion mobile application. For example, a dot is displayed on the screen which the user would be able to move up and down when inhaling and exhaling respectively (e.g. as shown in FIG. 4). Some examples of breathing exercises include the square-breathing mode 308. Square-breathing mode 308 can be used to implement square-breathing exercises. Square-breathing exercises can be used to heighten performance and concentration while also being a powerful stress reliever. In a square-breathing exercises, the user can inhale for a specified number of seconds and then hold the user's breath at this position for a specified period. After the breath hold is complete, the user can then exhale for a specified number of seconds and again hold this breath at end of the exhale position. FIG. 4 illustrate a series of example screen shots 400 of a square-breathing mode user interface, according to some embodiments. Screen shots 400 illustrate a square-breathing exercise visualization as displayed by the mobile-device application. The green dot can move up the curve when the user is inhaling and as the user holds the breath at that position the green dot follows the straight line. Then when they exhale the green dot moves down the curve and the same pattern follows. The mobile-device application can also play audio of soothing sounds (e.g. the sound of a waterfall, rainforest, ocean waves, etc.).

A virtual reality mode 308 can be implemented. In this mode, breathing exercises can be visualized in a 3D virtual space. Various meditation environments can also be displayed. These can include various famous temples, meditation halls, etc. from around the world. These can include various natural environments. Elements of the virtual environment can be based on the user's breathing patterns and/or other biosensor-derived patterns (e.g. hear rate can be synchronized with rain, waves can diminish in size and intensity with an increase in user relaxation, etc.). With the help of 3D animations in a virtual reality application the user would be able to perform breathing exercises.

A road-rage detection mode 310 can be implemented. Road-rage detection mode 310 can use machine-learning models and AI analysis methods on data from, inter alia: the accelerometer and gyroscope data, heart rate data and breathing volume data. The road-rage mode 310 can also obtain user context data to determine when a user is driving a vehicle (e.g. GPS data from the mobile-device, etc.). Sudden swings in the biosensor data can be used to determine when the user is experiencing road rage. Road-rage detection mode 310 can use haptic feedback and other feedback data (e.g. audio alerts, etc.) to remind the user to take deep breaths to reduce stress. Feedback can be audio feedback (e.g. an alert sound, a pre-recorded voice-based meditation guidance feedback, haptic feedback, visual cues/instruction (e.g. text, symbolic feedback, etc.), etc.). Road-rage detection mode 310 can detect when the emotional state of the user normalizes and alert the user as well.

Example Systems

FIG. 5 illustrates an example system 500 for implementing a breath monitoring and feedback device, according to some embodiments. System 500 can include computer/cellular/data networks 502. These can include, inter alia: the Internet, LANs, Bluetooth, Wi-Fi, etc. Breath monitoring and feedback device 502 can be communicatively coupled with mobile device 506. Mobile device 506 can include a breath monitoring and feedback application. Breath monitoring and feedback application can obtain data from the breath monitoring and feedback device 504. Breath monitoring and feedback application can use this data to provide feedback signals to the user. Breath monitoring and feedback application can display meditation exercises. Breath monitoring and feedback application can send/receive information from breath analysis and meditation guidance server(s) 508. Breath analysis and meditation guidance server(s) 508 can implement machine-learning analytics on user data. Breath analysis and meditation guidance server(s) 508 can store data in data store 512. Breath analysis and meditation guidance server(s) 508 can communicate with third-party system 510. Third-party systems 510 can be used to obtain various services such as, inter alia: search engines, database mangers, video editors, meditation databases, physician offices, medical study providers, etc. Breath analysis and meditation guidance server(s) 508 can include a store of meditation exercises that a user can access. Breath analysis and meditation guidance server(s) 508 can provide various e-payment services for purchasing meditation exercises. Breath analysis and meditation guidance server(s) 508 can provide a dashboard for meditation teachers to upload meditation sessions, exercises and the like. Breath analysis and meditation guidance server(s) 508 can format and render user data for display on a breath monitoring and feedback application. Breath analysis and meditation guidance server(s) 508 can implement various aspects of processes 100, 200 and 300.

FIG. 6 illustrate an example images of various aspects of a breath monitoring and feedback device 600, according to some embodiments. Breath monitoring and feedback device can be worn around the user's chest. Breath monitoring and feedback device 600 can include various Wi-Fi, Bluetooth, etc. systems for communicating with a local mobile device. Breath monitoring and feedback device can include a breathing-volume monitor system as shown in FIG. 6. Breath monitoring and feedback device 600 can use a strain gauge 604 on a load cell. Adjustable straps 606 are attached to the load cell 602 that wraps around the diaphragm area of a human body. The load cell 602 can initially be calibrated using the circumference of the diaphragm area at the resting state. Then, as that area expands (e.g. indicating inhaling) or contracts (e.g. indicating exhaling) the change of resistance in the load cell 602 is correlated to various states of breathing which are normal, shallow, or deep breathing. The accelerometer 616 and gyroscope 610 are used to detect the posture and the state of movement of the human body. The heart rate sensor 612 obtains the current heart rate of the attached human body. The values of posture, heart rate, and/or the breathing volume are used to detect the current emotional state of the human body wearing the sensor. These values can then be transmitted in substantially real-time over BLUETOOTH® system 608 (e.g. and/or other a wireless technology standard used for exchanging data between fixed and mobile devices over short distances using short-wavelength UHF radio waves in the industrial, scientific and medical radio bands, from 2.402 GHz to 2.480 GHz, and building personal area networks (PANS)) to a paired device (e.g. a mobile device). The paired device can then upload this data to a secure backend database service (e.g. as shown in FIG. 5 supra). With the help of a companion mobile application operative in the paired device, the user review the data related to their breathing volume in real time. This mobile-device application can provide various options to the user to various. The mobile-device application can also display/provide different breathing exercises. The user can view an historical breathing pattern during different periods throughout the day.

FIG. 7 illustrates an example use of a breath monitoring and feedback device 600 on a user 700, according to some embodiments. User 700 wears breath monitoring and feedback device 600. Breath monitoring and feedback device 600 can provide haptic signal 702 to user 700. Breath monitoring and feedback device 600 can use BLUETOOTH® system 608 to send a local area wireless signal 704 to mobile device 706. Mobile device 706 can include breath monitoring and feedback application. Mobile device 706 can display feedback 708 to the user as well.

Additional Systems and Architecture

FIG. 8 depicts an exemplary computing system 800 that can be configured to perform any one of the processes provided herein. In this context, computing system1 700 may include, for example, a processor, memory, storage, and I/O devices (e.g., monitor, keyboard, disk drive, Internet connection, etc.). However, computing system 800 may include circuitry or other specialized hardware for carrying out some or all aspects of the processes. In some operational settings, computing system 800 may be configured as a system that includes one or more units, each of which is configured to carry out some aspects of the processes either in software, hardware, or some combination thereof.

FIG. 8 depicts computing system 800 with a number of components that may be used to perform any of the processes described herein. The main system 802 includes a motherboard 804 having an I/O section 806, one or more central processing units (CPU) 808, and a memory section 810, which may have a flash memory card 812 related to it. The I/O section 806 can be connected to a display 814, a keyboard and/or other user input (not shown), a disk storage unit 816, and a media drive unit 818. The media drive unit 818 can read/write a computer-readable medium 820, which can contain programs 822 and/or data. Computing system 800 can include a web browser. Moreover, it is noted that computing system 800 can be configured to include additional systems in order to fulfill various functionalities. Computing system 800 can communicate with other computing devices based on various computer communication protocols such a Wi-Fi, Bluetooth® (and/or other standards for exchanging data over short distances includes those using short-wavelength radio transmissions), USB, Ethernet, cellular, an ultrasonic local area communication protocol, etc.

FIG. 9 illustrates an example time-series graph 900 of a user's breath measurements, according to some embodiments. Graph 900 shows a record a user's breathing rate for a period of twenty-four hours (24 hrs). It is noted that other graphs can show other time periods. It is further noted that a normal breathing rate for adults in a relaxed state is between twelve (12) and twenty-five (25) breaths-per-minute. A breathing rate over twenty-five (25) indicates the user is in a stressful state. Here in the graph 900, it is shown that the user's breathing rate was over twenty-five (25) between eleven thirty (11.30) am and twelve thirty (12.30) am (e.g. highlighted in the graph). The breathing rate can be captured by the device along with other parameters (e.g. heart rate, etc.) and use haptic feedback to inform the user to control of there breathing rate and ease out of the stressed state of mind. This can help the user in improving both there physical and mental health.

It is noted that breathing exercise can benefit, including but not limited to: help a user relax; lower the harmful effects of the stress hormone cortisol on your body; lower a user's heart rate, lower a user's blood pressure; help a user cope with the symptoms of post-traumatic stress disorder (PTSD); improve a user's core muscle stability; improve a user's body's ability to tolerate intense exercise; lower a user's chances of injuring or wearing out your muscles; slow a user's rate of breathing so that it expends less energy; etc.

### CONCLUSION

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g., embodied in a machine-readable medium).

In addition, it will be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed as new and desired to be protected by Letters Patent of the United States is:

1. A computerized method of a breath monitoring and feedback application comprising:
providing a breath monitoring and feedback application in a mobile device;
determining a user's breath state;
providing a means to monitor a user breath;
communicating a user breath statistics to the breath monitoring and feedback application;
determining a user's emotional state; and
with the breath monitoring and feedback application, communicating an application mediation technique to the user, wherein the application mediation technique is selected based on the user's breath state and the user's emotional state.

2. The computerized method of claim 1, wherein the step of determining the user's breath state comprises:
attaching a strain gauge to a load cell; and
strapping the strain gauge and the load cell around a user's diaphragm.

3. The computerized method of claim 1, wherein the means to monitor a user breath comprises the strain gauge attached to the load cell.

4. The computerized method of claim 2, wherein the step of determining the user's breath state comprises:
calibrating the loadcell using a circumference of the user's diaphragm at a resting state; and
as user breaths in and out, measuring a displacement force on the load cell.

5. The computerized method of claim 4 further comprising:
coupling, via a local area network, an accelerometer, a gyroscope, and a heartrate monitor to the breath monitoring and feedback application, wherein the accelerometer, the gyroscope and the heartrate monitor are worn by the user while the user is performing a breathing exercise.

6. The computerized method of claim 5, wherein the user's emotional state is determined by:
using a set of data from the accelerometer and the gyroscope to determine a user's posture state.

7. The computerized method of claim 6, wherein the user's emotional state is determined by:
using another set of data from the heartrate monitor to determine a user's heart rate.

8. The computerized method of claim 7, wherein the user's emotional state is determined by:
based on the set of data from the accelerometer and the gyroscope and the other set of data from the heartrate monitor, determining the emotional state of the user.

9. The computerized method of claim 8 further comprising:
utilizing a haptic motor worn by the user to provide a haptic signal to the user to take a deeper breath and maintain proper posture.

10. The computerized method of claim 9, wherein the breath monitoring and feedback application in the mobile device displays a graphical representation of the user's breath state as a function of time on the mobile device.

11. The computerized method of claim 10, wherein the graphical representation of the user's breath state and the user breath statistics comprises a square-breathing mode graphical representation.

12. A computer system of a breath monitoring and feedback application comprising:
a processor;
a memory containing instructions when executed on the processor, causes the processor to perform operations that:
provide a breath monitoring and feedback application in a mobile device;
determine a user's breath state;
provide a means to monitor a user breath;
communicate a user breath statistics to the breath monitoring and feedback application;
determine a user's emotional state; and
with the breath monitoring and feedback application, communicate an application mediation technique to the user, wherein the application mediation technique is selected based on the user's breath state and the user's emotional state.

13. The computerized system of claim 12, wherein the means to monitor the user's breath comprises:
a strain gauge attached to a load cell; and
wherein the strain gauge attached to the load cell includes a strap that is strapped around a user's diaphragm.

14. The computerized system of claim 13, wherein memory containing instructions when executed on the processor, causes the processor to perform operations that:
calibrate the loadcell using a circumference of the user's diaphragm at a resting state; and
as user breaths in and out, measuring a displacement force on the load cell.

15. The computerized system of claim 14, wherein memory containing instructions when executed on the processor, causes the processor to perform operations that:
couple, via a local area network, an accelerometer, a gyroscope, and a heartrate monitor to the breath monitoring and feedback application, wherein the accelerometer, the gyroscope and the heartrate monitor are worn by the user while the user is performing a breathing exercise.

16. The computerized system of claim 15, wherein the user's emotional state is determined by using a set of data from the accelerometer and the gyroscope to determine a user's posture state.

17. The computerized system of claim 16, wherein the user's emotional state is determined by using another set of data from the heartrate monitor to determine a user's heart rate.

18. The computerized system of claim 17,
wherein when the user is performing a breathing exercise the means to monitor a user breath tracks a number of inhalations and exhalations as the breath rate and provides a breath rate information to the user as a form of feedback;

19. The computerized system of claim 18,
wherein the means to monitor a user breath tracks a number of seconds the users is taking to inhale and provides an inhale breath time information to the user in the form of feedback, and
wherein the means to monitor a user breath tracks a number of seconds the users is taking to exhale and provides an exhale breath time information to the user in the form of feedback.

20. The computerized system of claim 18,
wherein the means to monitor a user breath tracks a depth of inhalations and exhalation the users is taking to inhale and exhale and provides an inhale and exhale depths of breath information to the user in the form of feedback, and
wherein based on the feedback of the feedback application provides breathing exercises with realtime audio feedback.
